SEQUENCE LISTING (8 sequences)

SEQ ID NO: 1 - DNA, 46 bp, Artificial Sequence
  Description of Artificial Sequenceoligonucleotide
ttaatacgactcactatagggagaatggtgagcaagggcgaggagc

SEQ ID NO: 2 - DNA, 45 bp, Artificial Sequence
  Description of Artificial Sequenceoligonucleotide
ttaatacgactcactatagggagagtacagctcgtccatgccgag

SEQ ID NO: 3 - DNA, 46 bp, Artificial Sequence
  Description of Artificial Sequenceoligonucleotide
ttaatacgactcactatagggagaatggggggttctcatcatcatc

SEQ ID NO: 4 - DNA, 45 bp, Artificial Sequence
  Description of Artificial Sequenceoligonuleotide
ttaatacgactcactatagggagactcaggtcaaattcagacggc

SEQ ID NO: 5 - DNA, 44 bp, Artificial Sequence
  Description of Artificial Sequenceoligonucleotide
ttaatacgactcactctcgggagaatgggccccggcccctgccg

SEQ ID NO: 6 - DNA, 45 bp, Artificial Sequence
  Description of Artificial Sequenceoligonucleotide
ttaatacgactcactatagggagagcctacctgcaccgtgtaccc

SEQ ID NO: 7 - DNA, 45 bp, Artificial Sequence
  Description of Artificial Sequenceoligonucleotide
ttaatacgactcactatcgggagaatgaatttgcaactggtttcc

SEQ ID NO: 8 - DNA, 43 bp, Artificial Sequence
  Description of Artificial Sequenceoligonucleotide
ttaatacgactcactctcgggagagccaccttctggagaatcc

(19) United States
(12) Patent Application Publication
(10) Pub. No.: US 2004/0235764 A1
(43) Pub. Date: Nov. 25, 2004

(54) Methods of inhibiting expression of a target gene in mammalian cells

(76) Inventors: Eric Billy, Chatenois (FR); Witold Filipowicz, Riehen (CH); Ulrich Mueller, Solana Beach, CA (US)

Correspondence Address: NOVARTIS, CORPORATE INTELLECTUAL PROPERTY, ONE HEALTH PLAZA 430/2, EAST HANOVER, NJ, 07936-1080, US

(21) Appl. No.: 10/469,447

(22) PCT Filed: Feb. 27, 2002
(86) PCT No.: PCT/EP02/02098

(30) Foreign Application Priority Data
Feb. 28, 2001 (GB) 0104948.5

(51) Int. Cl. A61K 48/00; C12N 15/85

(52) U.S. Cl. 514/44; 435/455

(57) ABSTRACT

A method is provided for inhibiting gene expression in a mammalian cell based on dsRNA, as well as construct useful for carrying out the invention and resulting cells and transgenic mammals.

### FIELD OF INVENTION

The present invention relates to the field of gene expression, in particular to the inhibition of gene expression in mammalian cells by double-stranded RNA. The double-stranded RNA technology has wide applications including determining gene function and developing therapeutic methods for treating diseases.

### BACKGROUND OF THE INVENTION

In the last few years, advances in nucleic acid chemistry and gene transfer have inspired new approaches to engineer specific inhibition of gene expression. Antisense technology has been the most commonly described approach in protocols to achieve gene-specific inhibition. For antisense strategies, single-stranded nucleic acid complementary to the messenger RNA of the gene of interest is introduced into the cell. Some difficulties with antisense-based approaches relate to delivery, stability, and dose requirements. In general, cells do not have an uptake mechanism for single-stranded nucleic acids, hence uptake of unmodified single-stranded material is extremely inefficient. While waiting for uptake into cells, the single-stranded material is subject to degradation. Because antisense technologies require that the single-stranded material acumulates at a relatively high concentration (at or above the concentration of endogenous mRNA), the amount required to be delivered is a major constraint on efficacy. As a consequence, much of the effort in developing antisense technology has been focused on the production of modified nucleic acids that are both stable to nuclease digestion and able to diffuse readily into cells. The use of antisense technologies for gene therapy or other whole-organism applications has been limited by the large amounts of oligonucleotide that need to be synthesized from non-natural analogues, the cost of such synthesis, and the difficulty even with high doses of maintaining a sufficiently concentrated and uniform pool of single-stranded material in each cell.

The use of triple helix technologies has also been proposed for inhibiting, gene expression, an approach that relies on the rare ability of certain nucleic acid populations to adopt a triple-stranded structure. Under physiological conditions, nucleic acids are virtually all single- or double-stranded, and rarely if ever form triple-stranded structures.

It has been known for some time, however, that certain simple purine- or pyrimidine-rich sequences can form a triple stranded molecule in vitro. Such structures are generally very transient under physiological conditions, so that simple delivery of unmodified nucleic acids designed to produce triple-stranded structures is not effective. As with antisense, development of triple-stranded technology for use in vivo has focused on the development of modified nucleic acids that would be more stable and more readily absorbed by cells in vivo. An additional goal in developing this technology has been to produce modified nucleic acids for which the formation of triple-stranded material proceeds effectively at physiological pH. A promising technology for achieving targeted gene silencing is based on double-stranded RNA (dsRNA) inducing a response called post-transcriptional gene silencing or RNA interference (RNAi). Double-stranded RNA has been introduced into a number of different species, including nematodes, fruit flies, Trypanosoma, fungi, plants. See for example, WO9932619. Some limited success has also been demonstrated in mammals, specifically in mouse oocytes and embryos. Introduction of the appropriate dsRNA inhibits gene expression in a sequence-dependent manner, an effect that has been used extensively in C. elegans and D. melanogaster as a genetic tool for studying gene function. For example, 00/01846 describes methods for characterizing gene function using dsRNA inhibition. However, dsRNA inhibition has been applied with little success in mammalian systems.

There have been reports in the literature attempting to achieve RNAi in mammalian systems. One successful report used dsRNA injected into preimplantation mouse oocytes to intefere with the function of reporter or endogenous genes (Wianny and Zemicka-Goetz (2000) Nature Cell Biology 2:70-75). A transgenic line of mice expressing a modified form of green fluorescent protein (GFP) was used to demonstrate that microinjection of dsRNA corresponding to modified GFP into zygotes specifically abrogated GFP expression. The silencing of GFP expression in the mouse embryo was observed until 6.5 days postimplantation. In addition, dsRNA for maternally expressed c-mos or zygotically expressed E-cadherin microinjected into the mouse oocyte or the zygote could be shown to induce a release from metaphase II arrest or perturbed development, respectively. Another report (Svoboda et al. (2000) Development 127, 4147-4156) describes the selective reduction of dormant maternal mRNAs (Mos and tissue plasminogen activator) after oocyte microinjection with specific dsRNA.

However, Caplen et al. found no evidence for specific, dsRNA-mediated gene silencing in mammalian tissue culture (Caplen et al., (2000) Gene 252:95-105). Transient co-transfection of reporter plasmid DNA with its corresponding dsRNA into human embryonic kidney cells (HEK 293) or baby hamster kidney cells BHK21 resulted in no effect (HEK 293) or in a non-specific decrease of the expression of the reporter gene (i.e., not dependent on the gene sequence). In addition, transfection of dsRNA into mouse fibroblast NIH3T3 cells transduced with a retrovirus expressing β-Gal, induced no detectable decrease in reporter gene expression. Conversely, Caplan et al. were able to visualize dsRNA-mediated gene silencing in cultured Drosophila cells.

Thus, although RNAi has been used extensively in non-mammalian systems, dsRNA inhibition has been applied with only limited success in mammalian systems. Although invertebrate systems provide valuable tools in analyzing biological function, a general system for evaluating gene function in mammalian systems would be advantageous, as well as allowing the development of therapies dependent on dsRNA inhibition.

### SUMMARY OF THE INVENTION

The present invention provides methods of inhibiting expression of a target gene by exposing a mammalian cell to a nucleic acid having at least a partially double-stranded ribonucleic acid and at least 60% sequence identity to the target gene, with the proviso that the mammalian cell is not a mouse zygote. The target gene can be an endogenous gene, an oncogene, a transgene, a viral gene or a gene derived from any infectious organism.

The methods of the invention can be used in any mammalian cell, including embryonic stem cells and cell lines and cancer cells. Protein kinase R and Rnase L may be advantageously inhibited in some mammalian cell lines by use of dsRNA specific for these genes in conjunction with dsRNA specific for a target gene.

The ribonucleic acid used for inhibition will have at least a partially double-stranded character, but may also be totally double-stranded. The RNA can be a single strand that is self-complementary or may comprise two or more separate complementary strands. The ribonucleic acid may also contain modified nucleotide residues. The RNA can be synthesized inside or outside of the cell. For example, it can be transcribed under the control of an endogenous promoter or a product of an expression vector in the cell. The expression vector may comprise a constitutive promoter, an inducible promoter or a tissue-specific promoter operably linked to the nucleic acid encoding the RNA. The expression vector may also comprise a promoter operably linked to a reporter gene, where the promoter is selected from the group consisting of a second promoter, a bi-directional promoter or a promoter driving a polycistronic message.

Thus, also provided by the present invention is an expression construct containing sequences encoding dsRNA molecules corresponding to PKR and one or more of RNAseL, 2′,5′ oligoadenylate cyclase, Mx protein and the nucleic acid encoding a dsRNA of interest (i.e., specific for a target gene of interest). Also provided is a kit containing reagents for inhibiting expression of a target gene in a cell, including a means for introduction of or expression of dsRNA specific for a target gene into a mammalian cell in an amount sufficient to inhibit expression of the target gene.

The present invention also provides mammalian cells, including embryonic stem (ES) cells, which exhibit gene silencing mediated by dsRNA, as well as a transgenic mammal, other than human, which are generated from the embryonic stem cells of the invention.

Also provided by the invention are methods for treating a disease or condition by administering to a subject ES cells exhibiting dsRNA-mediated gene inhibition or ds RNA.

Also provided are methods of assigning function to a DNA sequence of unknown function and methods of identifying DNA responsible for conferring a particular phenotype in a cell, by administering dsRNA to a cell, which methods are amenable to high through put technologies.

### DETAILED DESCRIPTION OF THE INVENTION

The present inventors have successfully used double stranded RNA to induce targeted inhibition of gene expression in mammalian cells. Prior to the present invention, specific dsRNA mediated gene silencing had not been successful in mammalian systems, other than by microinjection of mouse oocytes and zygotes. Thus, in its broadest aspect, the present invention provides a method of inhibiting expression of a target gene by exposing a mammalian cell to a nucleic acid of at least partially double-stranded character in an amount sufficient to inhibit expression of the gene in the cell.

The nucleic acid will typically be a ribonucleic acid (RNA) that in double-stranded form has at least 60%, preferably at least 80%, more preferably at least 90%-95% or most preferably 100% sequence identity to a portion of a target gene of interest. Preferred RNA molecules for inhibition comprise sequences identical to a portion of the target gene over at least 15 consecutive bases, preferably at least 20 consecutive bases, more preferably at least 25 consecutive bases. Nevertheless, RNA sequences with insertions, deletions, and single point mutations relative to the target sequence can also be designed to be effective for inhibition, allowing sequence variations that might be expected due to genetic mutation, polymorphism, or evolutionary divergence to be targeted. Gene expression is inhibited in a sequence-specific manner in that nucleotide sequences corresponding to the duplex region of the RNA are targeted for inhibition.

For ease of chemical synthesis and administration, the RNA is preferably as short as possible while maintaining specificity for its target gene. As is apparent to one of ordinary skill in the art, 18-20 identical, contiguous nucleotides are typically sufficient to achieve specificity for a particular human sequence The double-stranded portion of the RNA molecule is therefore typically at least 18-25 nucleotides in length, typically 18-20 nucleotides in length for use in human cells, optionally including one or preferably two 3′ overhangs. The overhangs are preferably only a few nucleotides in length to avoid non-specific interactions and typically are 1-10 nucleotides in length, preferably just 2-3 nucleotides in length.

Sequence identity may be optimized by sequence comparison and alignment algorithms known in the art (see Gribskov and Devereux, Sequence Analysis Primer, Stockton Press, 1991, and references cited therein) and calculating the percent difference between the nucleotide sequences by, for example, the Smith-Waterman algorithm as implemented in the BESTFIT software program using default parameters (e.g., University of Wisconsin Genetic Computing Group). Alternatively, for longer sequences, the duplex region of the RNA may be defined functionally as a nucleotide sequence that is capable of hybridizing with a portion of the target gene transcript (e.g., 400 mM NaCl, 40 mM PIPES pH 6.4, 1 mM EDTA, 50 C or 70 C hybridization for 12-16 hours; followed by washing). The length of the nucleic acid used to determine the degree of sequence identity depends on the length of the double-stranded portion of the RNA and therefore will be at least 18, at least 20, 25 or more bases in length. The RNA sequence is preferably chosen to have identity with exon sequences (in particular, mRNA sequences of the target gene).

The double-stranded RNA can be formed by a self-complementary RNA strand (such as a transcript having an inverted repeat), or by two or more complementary RNA strands. RNA duplex formation may be initiated either inside or outside the cell. The RNA is introduced in an amount that allows delivery of at least one copy per cell, preferably at least 5, 10, 100, 500 or 1000 copies per cell, depending on the application. The present inventors have demonstrated that dsRNA-meditated inhibition of a target gene can be dose dependent and therefore the amount introduced is dependent on the desired effect and can be easily determined empirically.

The nucleic acid may comprise nucleotides or linkages other than those that occur naturally in ribonucleic acid, for example, to stabilize the dsRNA from degradation, especially when RNA is delivered to a cell and not produced by the cell. Thus, one can employ oligoribonucleotides or oligonucleotides that comprise one or more modified (i.e., synthetic or non-naturally occurring) nucleotides. Usually, nucleotide monomers in a nucleic acid are linked by phosphodiester bonds or analogues thereof. Analogues of phosphodiester linkages include phosphorothioate, phosphorodithioate, phosphoroselenoate, phosphorodiselenoate, phosphoroanilothioate, phosphoranilidate, phosphoramidate, peptide, and the like linkages. Those of skill in the art will recognize that the reagents employed are commercially available or, in the case of the oligonucleotides, can be prepared using commercially available instrumentation. Preferably the duplex RNA will comprise ribonucleotide units or other nucleotide units that allow appropriate processing by the cell and efficient inhibition of the target gene.

Now that the present inventors have developed methodology to implement dsRNAi effectively in mammalian cell culture, it is expected that dsRNAi can be applied to a number of mammalian cell types for targeting inhibition of a gene of interest. Thus, the cell may be cells from the inner cell mass, extraembryonic ectoderm or embryonic stem cells, totipotent or pluripotent, dividing or non-dividing, parenchyma or epithelium, immortalized or transformed, or the like. The cell may be a stem cell or a differentiated cell. Cell types that are differentiated include without limitation adipocytes, fibroblasts, myocytes, cardiomyocytes, endothelium, dendritic cells, neurons, glia, mast cells, blood cells and leukocytes (e.g., erythrocytes, megakaryotes, lymphocytes, such as B, T and natural killer cells, macrophages, neutrophils, eosinophils, basophils, platelets, granulocytes), epithelial cells, keratinocytes, chondrocytes, osteoblasts, osteoclasts, hepatocytes, and cells of the endocrine or exocrine glands, as well as sensory cells.

As described above, RNA interference has been demonstrated in mouse oocytes (haploid genome) and zygotes (post-fertilization) and such embodiments are not meant to be encompassed by the present invention. The term “renewable”, as used herein refers to mammalian cells and their progeny, which are capable of dividing to produce replicas of themselves, either in cell culture or in vivo. Although the Examples below demonstrate the invention using embryonic cell lines and embryonic stem cells, the invention is not so limited, as it is now expected that the methods of the invention can be applied to various mammalian cell lines.

In some cases, it might be advantageous to inhibit the protein kinase R (PKR-NCBI Accession no. XM 002661) or/and the interferon pathway that can result in apoptosis, (Lee, 1994; Lee, 1996), pathways that are often active in differentiated cells. This can now be easily achieved by introducing an inhibitory RNA duplex specific for inhibition of PKR and/or RNAse L, optionally also inhibiting Mx protein (Fuji et al. (1999) Virus Res. 65:175-185). One of ordinary skill in the art will recognize that RNAse L (NCBI Accession no. AF281045 or human equivalent) can be inhibited indirectly by inhibiting one or more 2′5′-oligoadenylate synthetase genes (2-5 AS; e.g., NCBI Accession No. NM 006187) responsible for activating RNAseL, for example using the dsRNAi methodology described herein. As will also be apparent to one of ordinary skill in the art, other methodologies can be used to inhibit the PKR and/or interferon pathways, such as the use of specific chemical inhibitors for PKR (e.g., chemical inhibitor of caspase 8, Gil and Esteban, 2000, Oncogene 19:3665-74) or RNase L, respectively.

The RNA may be synthesized either in vivo or in vitro. Endogenous RNA polymerase of the cell may mediate transcription in vivo, or cloned RNA polymerase can be used for transcription in vivo or in vitro. For transcription from a transgene in vivo, an expression construct comprising at least one regulatory region (e.g., promoter, enhancer, silencer, splice donor and acceptor and polyadenylation signal) operably linked to the DNA coding for the desired RNA transcript(s) may be used to transcribe the RNA strand (or strands). The promoter can be of almost any origin. Preferred are promoters that are active in the chosen host cells like the SV40, beta-actin, metallothionein, T7, polyhedrin and cytomegalovirus promoters. The promoter can be a constitutive promoter, an inducible promoter or a tissue-specific promoter, for example, allowing inhibition to be targeted to an organ or cell type; or transcription to be induced upon stimulation of an environmental condition (e.g., infection, stress, temperature, chemical inducers); and/or engineering transcription at a developmental stage or age. Alternatively, a knock-in construct can be used to transcribe the nucleic acid of interest under the control of an endogenous promoter, as is known in the art. Modified or unmodified RNA can also be chemically or enzymatically synthesized by manual or automated reactions. The RNA may be synthesized by a cellular RNA polymerase or a bacteriophage RNA polymerase (e.g., T3, T7, SP6).

Expression vectors may also include sequences allowing for their autonomous replication within the host cell, sequences that encode genetic traits that allow cells containing the vectors to be selected, and sequences that increase the efficiency with which the RNA is transcribed. Stable long-term vectors may be maintained as freely replicating entities by using regulatory elements of viruses. Cell lines may also be produced which have integrated the vector into the genomic DNA and in this manner the transcript(s) is/are produced on a continuous basis.

Thus, an expression vector can further include additional sequences operably linked to a promoter, such as a reporter gene (e.g., fluorescent proteins, e.g., green fluorescent protein, β-galactosidase, alkaline phosphatase, luciferase, CAT, selective gene markers that facilitate the selection of transformants due to the phenotypic expression of the marker gene (e.g., those expressing antibiotic resistance or, in the case of auxotrophic host mutants, genes which complement host lesions), or other useful sequences, such as those encoding dsRNA corresponding to PKR, RNAse L, 2-5 AS and proteins of the Mx family.

Nucleic acids can be introduced into a cell by various standard methods in genetic engineering, including physical methods, for example, by injection of a solution containing the nucleics acid, bombardement by particles covered by the nucleic acid, soaking the cell or organism in a solution of the nucleic acid, or electroporation of cell membranes in the presence of the nucleic acid. A particularly preferred method for delivering nucleic acids is the use of electroporation. Alternatively, a viral construct accomplishes both efficient introduction of an expression construct into a cell and transcription of RNA encoded by the expression construct. Other methods known in the art for introducing nucleic acids to cells may also be used, as is apparent to the artisan, such as lipid-mediated delivery systems, chemical mediated transport, such as calcium phosphate transfection, DEAE-dextran transfection, and the like. Thus, the RNA may be introduced along with components that perform one or more of the following activities: enhance RNA uptake by the cell, promote annealing of the duplex strands, stabilize the annealed strands, inhibit RnaseL, PKR or protein Mx or any other protein that is responsible for preventing gene specific effects of dsRNA, or otherwise increase inhibition or prevent disease/pathology. Transfected host cells can be cultured by standard methods in cell culture.

The effect of dsRNA on gene expression will typically result in expression of the target gene being inhibited by at least 10%, 33%, 50%, 90%, 95%, 99% or more when compared to a cell not treated according to the present invention. Lower doses of administered material, lower concentrations of dsRNA in the cell and/or longer times after administration of dsRNA may result in inhibition at a lower level and/or in a smaller fraction of cells (e.g., at least 10%, 20%, 50%, 75%, 90%, or 95% of targeted cells). However, it is within the skill of the art to adapt conditions to provide the desired result. Quantitation of gene expression can be established by assessing the amount of the targeted gene product in the cell. For example, any mRNA transcribed from the target gene may be detected with a hybridization probe, or RT-PCR based methodologies, or translated polypeptide may be detected with an antibody raised against the encoded polypeptide.

As disclosed herein, the present invention is not limited to any type of target gene or nucleotide sequence. For example, the target gene can be a cellular gene, an endogenous gene, an oncogene, a transgene, or a viral gene including translated and non-translated RNAs. Preferably, the target gene is a cellular gene as exemplified by the following classes of possible target genes, which are listed for illustrative purposes only and are not to be interpreted as limiting: transcription factors and developmental genes (e.g., adhesion molecules, cyclin kinase inhibitors, Wnt family members, Pax family members, Winged helix family members, Hox family members, cytokines/lymphokines and their receptors, growth/differentiation factors and their receptors, neurotransmitters and their receptors); oncogenes (e.g., ABLI, BCLI, BCL2, BCL6, CBFA2, CBL, CSFIR, ERBA, ERBB, ERBB2, ETSI, ETV6, FGR, FOS, FYN, HCR, HRAS, JUN, KRAS, LCK, LYN, MDM2, MLL, MYB, MYC, MYCLI, MYCN, NRAS, PIMI, PML, RET, SKP2, SRC, TALI, TCL3, and YES); tumor suppressor genes (e.g., APC, BRAI, BRCA2, CTMP, MADH4, MCC, NFI, NF2, RBI, TP53, and WTI); and enzymes (e.g., ACP desaturases and hydroxylases, ADP-glucose pyrophorylases, ATPases, alcohol dehydrogenases, amylases, amyloglucosidases, catalases, cyclooxygenases, decarboxylases, dextrinases, DNA and RNA polymerases, galactosidases, glucose oxidases, GTPases, helicases, integrases, insulinases, invertases, isomerases, kinases, lactases, lipases, lipoxygenases, lysozymes, peroxidases, phosphatases, phospholipases, phosphorylases, proteinases and peptidases, recombinases, reverse transcriptases, telomerase, including RNA and/or protein components, and topoisomerases).

By inhibiting enzymes at one or more points in a metabolic pathway or genes involved in pathogenesis, the effect may be enhanced: each activity will be affected and the effects may be magnified by targeting multiple different components. Metabolism may also be manipulated by inhibiting feedback control in the pathway or production of unwanted metabolic byproducts.

The dsRNA can therefore be used in cells in vitro or ex vivo and then subsequently placed into an animal for therapeutic effect, or used for direct treatment by in vivo administration of the dsRNA. A method of gene therapy can therefore be envisioned, typically by introducing dsRNA specific for a target gene into a cell together with means for inhibiting the Pkr and RNAseL pathways. Any target gene known to cause the disease or condition needing treatment can be used. For example, tumor cells can be targeted using homing viral vectors, tumor-specific promoters or by designing dsRNA molecules effective in inhibiting tumor-specific genes (e.g., telomerase) and oncogenes. Treatment includes amelioration or avoidance of any symptom associated with the disease or clinical indication associated with the pathology, and this may include prophylactic therapy. A further preferred embodiment relates to administering to a subject ES cells treated with dsRNA to inhibit a desired target gene.

A gene derived from a pathogen (i.e., non-cellular gene) may also be targeted for inhibition. For example, the gene could cause immunosuppression of the host directly or be essential for replication of the pathogen, transmission of the pathogen, or maintenance of the infection. Cells at risk for infection by a pathogen or already infected cells, such as cells infected by human immunodeficiency virus (HIV) infections, influenza infections, malaria, hepatitis, plasmodium, cytomegalovirus, herpes simplex virus, and foot and mouth disease virus may be targeted for treatment by introduction of RNA according to the invention. The target gene might be a pathogen or host gene responsible for entry of a pathogen into its host, drug metabolism by the pathogen or host, replication or integration of the pathogen's genome, establishment or spread of an infection in the host, or assembly of the next generation of pathogen. Methods of prophylaxis (i.e., prevention or decreased risk of infection), as well as reduction in the frequency or severity of symptoms associated with infection, can be envisioned.

The present invention also provides methods of identifying gene function in an organism comprising the use of double-stranded RNA to inhibit the activity of a target gene of previously unknown (or unrecognized) function. Instead of the time consuming and laborious isolation of mutants by traditional genetic screening, functional genomics would envision determining the function of uncharacterized genes by employing the invention to reduce the amount and/or alter the timing of target gene activity. The invention could be used in determining potential targets for pharmaceuticals, understanding normal and pathological events associated with development, determining signaling pathways responsible for postnatal development/aging, and the like. The nucleotide sequence information acquired from genomic and expressed gene sources, including the human genome, can be coupled with the invention to determine gene function in mammalian systems, in particular in human cell culture systems. Putative open reading frames can be determined from nucleotide sequences available in databases using computer-aided searching techniques, as is apparent to one of ordinary skill in the art. Such techniques may take into consideration preferred codon usage in mammalian systems, and searches of sequence databases for related gene products (possibly including non-mammalian sequences) or homologies with genes of known function.

Thus, in one aspect of the invention, a method is provided for assigning function to a DNA sequence, whereby a mammalian cell is exposed to a nucleic acid having at least a partially double-stranded ribonucleic acid characteristics as described above and having at least 60% sequence identity (preferably 100% identity) to a desired DNA sequence of unassigned function, in an amount sufficient to inhibit gene expression of the cellular homologue of the desired DNA sequence, identifying a phenotype of the mammalian cell compared to wild type, and assigning the phenotype to the desired nucleic acid. It should be noted that preferred characteristics of the dsRNA, cells, other materials and conditions are those described above.

A simple assay would be to inhibit gene expression according to the partial sequence available from an expressed sequence tag (EST). Functional alterations in growth, development, metabolism, disease resistance, or other biological processes would be indicative of the normal role of the EST's gene product. If database screening finds a region of homology with a protein of known function, a more specific biochemical test based on that function can be used to test for the function of the EST sequence (or inhibition thereof).

The ease with which RNA can be introduced into an intact mammalian cell containing the target gene allows the present invention to be used in high throughput screening (HTS). For example, duplex RNA can be produced by an amplification reaction using primers flanking the inserts of any gene library derived from the target cell. Inserts may be derived from genomic DNA or mRNA (e.g., cDNA and cRNA). Individual clones (or pools thereof) from the library can be replicated and then isolated in separate rections, but preferably the library is maintained in individual rection vessels (e.g., a 96-well microtiter plate) to minimize the number of steps required to practice the invention and to allow automation of the process. Solutions containing duplex RNAs that are capable of inhibiting the different expressed genes can be placed into individual wells positioned on a microtiter plate as an ordered array, and intact cells in each well can be assayed for any changes or modifications in behavior or development due to inhibition of target gene activity or by proteomic, genomics and standard molecular biology techniques. Alternatively, the duplex RNA can be produced by in vivo or in vitro transcription from an expression construct used to produce the library. The construct can be replicated as individual clones of the library and transcribed to produce the RNA; each clone can then be introduced into the cell containing the target gene. The function of the target gene can be assayed from the effects it has on the cell when gene activity is inhibited. This screening is particularly amenable to tissue culture cells derived from mammals.

Advantageously, a cell that produces a colorimetric, fluorogenic, or luminescent signal in response to a regulated promoter (e.g., transfected with a reporter gene construct) can be assayed in a high throughput format to identify DNA-binding proteins that regulate the promoter. In the assay's simplest form, inhibition of a negative regulator results in an increase of the signal and inhibition of a positive regulator results in a decrease of the signal.

Thus, a method is provided for identifying DNA responsible for conferring a particular phenotype in a cell by introducing into one or more mammalian cells a cDNA or genomic library of the DNA of a desired cell in a suitable vector in an orientation relative to a promoter(s) capable of initiating transcription of the cDNA or DNA to provide double stranded (ds) RNA, cloning the cells and correlating a particular cell phenotype with a particular DNA or cDNA fragment from the library.

The present invention may be useful in allowing the inhibition of essential genes. Such genes may be required for cell or organism viability at particular stages of development or cellular compartments. The functional equivalent of conditional mutations may be produced by inhibiting activity of the target gene when or where it is not required for viability. The invention allows addition or expression of RNA at specific times of development and locations in the organism without introducing permanent mutations into the target genome.

If alternative splicing produced a family of transcripts that were distinguished by usage of charateristic exons, the present invention can target inhibition through the appropriate exons to specifically inhibit or to distinguish among the functions of family members. For example, a hormone that contained an alternatively spliced transmembrane domain may be expressed in both membrane bound and secreted forms. Instead of isolating a nonsense mutation that terminates translation before the transmembrane domain, the functional consequences of having only secreted hormone can be determined according to the invention by targeting the exon containing the transmembrane domain (i.e., the sequence encoding the transmembrane domain) and thereby inhibiting expression of membrane-bound hormone.

In a further aspect of the invention, stable cell lines, such as embryonic cell lines or cell lines comprising an expression cassette optionally allowing expression of dsRNA specific for PKR and RnaseL, can be used to evaluate specific transcription patterns in the presence and absence of dsRNA specific for a target gene. Northern mRNA analysis or microarray analysis can be used to determine whether a target gene is expressed in any given cell line. If dsRNA specific for a target gene is shown to affect expression of a particular gene, biochemical assays can then be used to confirm a direct or indirect relationship between the target gene and the gene with altered expression. Variations of this system can be envisioned by one of skill in the art, for example, by following expression changes in the presence of a ds RNA specific for a target gene versus a non-specific ds RNA, or following changes in expression in a cell line expressing the gene of interest vs in a cell line not expressing the gene of interest. The present invention also provides for the production of transgenic non-human animal models for the study of gene function, for the screening of candidate pharmaceutical compounds, and for the evaluation of potential therapeutic interventions. Animal species which are suitable for use in the animal models of the present invention include, but are not limited to rats, mice, hamsters, guinea pigs, chickens, rabbits, dogs, cats, goats, sheep, pigs, and non-human primates such as monkeys and chimpanzees. For initial studies, transgenic mice and rats are highly desirable due to their relative ease of maintenance and shorter life spans. For longer term studies, non-human primates may be desired.

To create a transgenic mouse with a loss-of-function phenotype, which is preferred, dsRNA can be induced in an embryonic stem cell in culture and used to generate a transgenic mouse. “Loss-of-function” is used in this disclosure to include hypomorph phenotypes, whether in transgenic animals or cells. In general, techniques of generating transgenic animals are widely accepted and practiced. A laboratory manual on the manipulation of the mouse embryo, for example, is available detailing standard laboratory techniques for the production of transgenic mice (Hogan et al., 1986). In a preferred method, embryonic stem cells, preferably stably transformed with a cassette expressing specific dsRNAs for PKR, RNaseL and a gene of interest, and expressing a reporter and/or selection marker, are selected for expansion in cell culture prior to implantation into a pseudo-pregnant female mouse. Similar methodologies can be used to use other transgenic animals, as is apparent to one of ordinary skill in the art.

The present invention also provides a kit comprising at least one reagent necessary to carry out the in vitro or in vivo introduction of dsRNA to test samples or subjects, or a construct for expression of dsRNA for inhibiting expression of a target gene in a mammalian cell. The kit comprises a means for introduction of a ribonucleic acid (RNA) into a mammalian cell in an amount sufficient to inhibit expression of the target gene, wherein the RNA has at least a partially double-stranded structure and sufficient nucleotide sequence identity as compared to a portion of the target gene to provide specificity, as described above. Preferred are kits comprising at least one expression vector capable of producing the dsRNA corresponding to at least a portion of the target gene of interest and dsRNA corresponding to at least a portion of PKR, RNAseL, protein Mx and/or 2-5 AS. Such a kit may also include instructions to allow a user of the kit to practice the invention.

The invention is further described, for the purposes of illustration only, in the following examples. Methods of molecular genetics, protein and peptide biochemistry and immunology referred to but not explicitly described in this disclosure are reported in the scientific literature and are well known to those skilled in the art.

### EXAMPLES

Example 1

Double-Stranded RNA-Mediated Silencing of an Exogenous Gene in Mouse Embryonic Stem Cells in Culture

This example demonstrates that dsRNA-mediated gene silencing can be induced in mammalian cultured cells. For convenience, green fluorescent protein (GFP) was chosen as an exogenous gene to study dsRNA interference (dsRNAi), because a fluorescence activated cell sorter (FACS) can be easily applied to sort cells expressing green fluorescent protein versus those that do not, e.g, those that exhibit gene silencing.

The effect of dsRNA on transgene GFP expression was addressed by co-transfecting mouse embryonic stem cells with a reporter eGFP- expressing plasmid, βact-eGFP (Ludin et al., Gene, 1996, Gene 173:101-11), together with dsRNA eGFP or dsRNA LacZ (a control sequence unrelated to GFP). The number of eGFP positive cells was determined by FACS analysis two and three days after co-transfection of the cells.

Preparation of dsRNA

The pβact-eGFP plasmid was used as a template in the Ambion T7 MegaScript kit for generating T7 promoter-tagged PCR products using previously described procedures (Fire, 1998, Nature; Dixon, 2000, PNAS). The T7-tagged PCR product was then used as template for in vitro transcription following the manufacturer's guidelines to generate eGFP RNA using TTAATACGACTCACTATAGGGAAATGGTGAGAAGGCGA GGAGC (SEQ ID NO:1) and TTAATACGACTCACTATAGGGAGAGTACAGCTCGTCC ATGCCGAG (SEQ ID NO:2) as primers. For LacZ ds RNA, plnd lacZ (commercially available construct encoding lacZ) was treated essentially as described above for βact-eGFP but using TTMTACGACTCACTATAGGGAGAAGGGGGGTTCTCATCATCATC (SEQ ID NO:3) and TTAATCGACTCACTATAGGGAGACTCAGGTCAAATTCAG ACGGC (SEQ ID NO:4) primers. RNA was precipitated with lithium chloride and then resuspended in nuclease-free water. RNA was then annealed by heating at 95° C. for 3 minutes, then transferring to 75° C., followed by slow cooling to room temperature (about 4 to 6 hours). The final concentration of dsRNA (approx. 700 bp long) was adjusted to 2-3mg/ml. The dsRNAs produce in this manner has a 3′ overhang corresponding to the T7 promoter sequence. Integrity and quality of the dsRNA was analyzed by electrophoresis on 1.5% agarose gels. Electrophoresis of control samples (dsDNA and ssRNA) showed that migration of dsRNA is similar to the corresponding dsDNA, but slower than the migration of the corresponding sense or antisense single stranded RNA. In addition, in contrast to ssRNA, the dsRNA was demonstrated to be resistant to RNase A and T1 treatments.

Cell Culture and Nucleic Acid Transfection

Mouse embryonic stem cells (commercially available, ES E-14 cells) were cultured in Dulbecco's Modified Eagle Medium (DMEM medium), 10% fetal bovine serum (FBS) supplemented with Na-pyruvate, L-Glutamine, non-essential amino acids [1:100 dilution of stock solution, Life Technologies] and leukemia inhibitory factor (LIF) (dilution 1:10000; Life Technologies) in tissue culture plates coated with inactivated-fibroblast feeder cells. ES cells were transfected with βoact-eGFP plasmid (30 μg) with or without dsRNA (30 μg) for eGFP or LacZ by electroporation under the following conditions: 10 million cells in 800 l of culture medium (described above), 250V, 500 μF. After electroporation, the cells were incubated for 10 minutes at room temperature before resuspending in DMEM medium (see above) and plating at a density ˜2×10₆ cells/10 cm plate. The medium was replaced every 24 hours.

The percentage of transfected and dsRNA co-transfected cells, as well as the in vivo dsRNA-mediated gene silencing of eGFP was assessed by measuring the level of green fluorescent protein expression by fluorescence activated cell sorter analysis (FACS, FacsCalibur, Becton Dickinson). Wild type cells and ppact-eGFP transfected cells were used to gate for forward scatter and side scatter; 10 000 or 25 000 events were captured per sample. The percentage of eGFP positive cells was determined by gating against wild type and ppact-eGFP transfected cells. The mean fluorescence was used as a measure of the relative intensity of fluorescence. Acquisition and analysis of the FACS data were performed using the CELLQuest Software (Becton Dickinson).

Two days post-transfection, about 30% of the mouse ES cells were eGFP positive following transfection with βact-eGFP. The co-transfection of dsRNA LacZ did not result in any alteration in the number of eGFP positive cells or in the level of mean fluorescence. In contrast, ES cells co-transfected with dsRNA eGFP, exhibit a strong reduction in the number of fluorescent cells (˜50%), correlating with an increase in eGFP negative cells compared to the control and the dsRNA LacZ co-transfected cells. In addition, the mean fluorescence of the remaining eGFP positive cells was also reduced by 40% up on co-transfection with dsRNA eGFP.

In Vivo Fluorescent Microscopy Analysis

The effect of dsRNA eGFP on the number of GFP positive cells and on the level of fluorescence was confirmed by in vivo fluorescent microscopy of growing ES cells. Cells were grown, after co-transfection at 37° C. with 5%CO₂, on an acid-washed coverslip in a 6-well plate. Cells were observed in DMEM Special non-autofluorescent media (Life Technologies, Basel) at 37° C. in purpose-built observation chambers (Life imaging Services, Olten, Switzerland) to avoid “cold-shock” to the cells when imaging, using a GFP-optimized filter set (ChromaTechnologies, Brattolboro, Vt.) installed on a Leica DM IRBE microscope. Illumination intensity was adjusted using neutral-density filters, images were taken with 1 second exposure time using a MicroMax cooled CCD camera (Princeton Instruments, Trenton, N.J.) and Metamorph 4.1.5 imaging software (Universal Imaging corporation, West Chester, Pa.).

For each sample, the fluorescence was recorded, measured using a software-based auto scale set-up that adjusts signal-to-noise automatically and then normalized to values obtained from a control sample corresponding to βact-eGFP transfected cells. Results from in vivo imaging two and three days post-transfection confirmed the results obtained from FACS analysis of cells co-transfected with dsRNA eGFP, an inhibitory effect being visualized only in the presence of dsRNA eGFP.

The level of eGFP protein in the cells is analyzed by Westem-blot using an anti-eGFP antibody to provide further evidence for a decrease of eGFP expression in dsRNA eGFP-containing cells.

These experiments demonstrate a substantial and specific decrease in both the number of positive eGFP cells and the relative eGFP fluorescence, which is associated with dsRNA eGFP-mediated gene silencing. For the first time, dsRNA is shown to be functional in mammalian cell culture and effective in inducing dsRNA-mediated gene silencing.

### Example 2

### dsRNA-Mediated Exogenous Gene Silencing in Embryonic Teratocarcinoma and Non-Embryonic Cell Lines in Culture

Although specific gene silencing by dsRNAi in ES cells could provide a tool to study genes involved in early steps of embryogenesis or to generate “loss-of-function” transgenic mice, ES cells require inactivated-fibroblast-coated plates for growth and are difficult to maintain in a non-differentiated state in culture for more than 4 to 5 passages. There would therefore be certain advantages in using a technically less-demanding cell type, for example, where the use of dsRNAi for studying gene function is envisioned. This example describes inducing dsRNA-mediated gene silencing in embryonic carcinoma cell lines, which are easier to work with and to propagate.

The effect of dsRNA on transgene GFP expression in cell lines was addressed by co-transfecting F9 and P19 embryonic carcinoma cell lines and the non-embryonic HeLa cells with a reporter eGFP- expressing plasmid, ppact-eGFP, together with dsRNA eGFP, dsRNA LacZ, ds RNA integrin a3 and P1 (control sequences unrelated to GFP).

The number of eGFP positive cells was analyzed by FACS two and three days after co-transfection of the cells.

Preparation of dsRNA

Ds RNAs were prepared essentially as described above in Example 1. The T7-tagged PCR products used for generating dsRNA for integrin α3, or integrin β1 were obtained by amplification of integrin α3 containing plasmid using TTAATACGACTCACTATAGG GAAATGGGCCCCGGCCCCTGCCG (SEQ ID NO:5) and TTAATACGACTCACTATA GGGAGAGCCTACCTGCACCGTGTACCC (SEQ ID NO:6) as primers and with integrin β1 encoding plasmid using TTAATACGACTCACTATAGGGAGAATGAATTTGCAACT GGTTTCC (SEQ ID NO:7) and TTAATACGACTCACTATAGGGAGAGCCACCTTCTGGAGAA TCC (SEQ ID NO:8) as primers.

Cell Culture and Nucleic Acid Transfection

F9 and P19 embryonic carcinoma cell lines were grown in DMEM medium+15% FBS and DMEM+10% FBS, respectively. HeLa cells were grown in DMEM+10% FBS and treated as P19 cells unless otherwise noted. F9 cells were maintained on 0.1% gelatin coated plates. F9 and P19 cells were passaged every 2 days to maintain exponential growth and their embryonic phenotype. F9 and P19 cells were split 24 hrs before transfection to ensure a high level of viable cells. F9 and P19 cells were transfected with βact-eGFP plasmid (30 μg) with or without dsRNA (30 μg) for eGFP, LacZ or integrinα3, or integrinβ1 by electroporation under the following conditions: 10 million cells in 800 μl of culture medium, 330V, 500 μF. After electroporation, the cells were incubated for 10 minutes at room temperature before resuspending in DMEM medium (see above) and plating at a density ˜2×10⁶ cells/10 cm plate. The medium was replaced every 24 hours.

The percentage of transfected and dsRNA co-transfected cells, as well as the in vivo dsRNA-mediated gene silencing of eGFP was assessed by measuring the level of green fluorescent protein expression by FACS, essentially as described above in Example 1. About 25% of the F9 or P19 cells transfected with pβact-GFP were found to be GFP positive.

In F9 and P19 cells, co-transfection of dsRNA eGFP with reporter βact-eGFP plasmid induced a strong and a dsRNA eGFP-specific reduction in both the number of eGFP-expressing cells (by over 70%) and the relative intensity of eGFP fluorescence (by 35-40%). In contrast, co-transfection of dsRNA-LacZ resulted in no alteration of either the number of GFP-positive cells or their mean fluorescence. In vivo fluorescent microscopy analysis was carried out essentially as described above in Example 1, except that the acid-washed coverslip was pre-coated with 0.1% gelatin for F9 cells. Both the in vivo imaging, Northern analysis and Westem-blot analysis confirmed the specific, inhibitory effect of dsRNA eGFP. The gene specificty of the dsRNAi effect on eGFP expression was also assessed in vivo in F9 and P19 cells using two other dsRNA corresponding to integrinα3, or integrinβ1. No effect on either the number of eGFP positive cells or on the level of fluorescence could be identified, again supporting a gene-specific effect in F9 and P19 cells.

In contrast, co-transfection of HeLa cells with pβact-eGFP and dsRNA corresponding to eGFP or LacZ induced a non-sequence specific gene silencing and cell death, as measured by the absence of eGFP fluorescent cells. Co-transfection of HeLa cells with the reporter ppact-eGFP plasmid and dsRNA is highly efficient since the results of FACS analysis showed the number of eGFP positive cells in dsRNA co-transfected experiments to be close to background (i.e., less than 1%), when compared to cells that received only the reporter plasmid.

The dose dependence of the dsRNA effect was investigated in P19 cells. Briefly, 30 μg of total dsRNA (30, 20, 10 or 0 μg eGFP dsRNA with 0, 10, 20, or 30 μg lacZ dsRNA, respectively) was introduced into P19 cells essentially as described above. The inhibitory effect on the number of GFP-positive cells and also on their mean fluorescence was demonstrated to be proportional to the amount of dsRNA-GFP used for co-transfection. The relative percentage of eGFP positive cells increased from 30, 50, 80 to 100% with decreasing eGFP dsRNA amounts.

Replacement of the GFP dsRNA described above having a 3′ overhang, with a blunt-end GFP dsRNA prepared by annealing anti-sense RNA with sense RNA prior to introduction into the cells resulted in a 50% inhibition compared to a 70% inhibition obtained with the dsRNA comprising an overhang.

Replacement of dsRNA-GFP with the same amount of a single-stranded GFP-specific RNA of either sense or antisense orientation resulted in no effect (sense RNA) or only 30% inhibition (antisense RNA) compared to 80% inhibition obtained with ds RNA.

Thus, the degree of inhibition can potentially be adjusted by controlling the amount of dsRNA, and by designing dsRNA to have blunt ends or an overhang, for example. Although electroporation was used as the method for transfection/co-transfection in the Examples above, the inventors have used various methods to introduce dsRNA into a cell to mediate gene silencing. For example, calcium phosphate, LipoFectamine 2000 and fugene6 have been used for F9 and P19 cells. Variable results were obtained although typically these different methods resulted in a lower number of transfected cells, making electroporation a preferred method for transfection.

In summary, this example demonstrates that specific, in vivo dsRNA-mediated gene silencing can be induced in mammalian embryonic cell lines. The ability to bring about dsRNA- mediated gene silencing in mammalian cell culture provides a new system allowing the elucidation of gene function and regulation.

### Example 3

### dsRNA-Mediated Gene Silencing of Endogenous Genes in Embryonic Teratocarcinoma Cell Lines

In this example, dsRNA is demonstrated to be effective in inhibitng endogenous gene expression. For illustrative purposes, dsRNAs corresponding to two receptor proteins, integrin α3 and integrin β1, were chosen so as to make use of a simple adhesion assay to monitor the presence or absence of the corresponding proteins on the cell surface. Integrin α3 is associated with integrin β1 and is responsible for the binding to the extracellular matrix molecule, laminin. Integrin β1 can interact with many different integrin α-types, resulting in binding specificity to other types of extracellular matrix molecules e.g., fibronectin (integrins α5β1, α8β1, αvβ1).

Using a polyclonal antibody against mouse integrin α3 and β1 and FACS analysis, we have shown the presence of these integrins on the surface of F9 cells as well as for P19 cells.

Embryonic F9 cells were co-transfected with pβact-eGFP reporter plasmid with either dsRNA LacZ (positive control), dsRNA eGFP (negative control), dsRNA α3 or dsRNA β1, essentially as described in Example 2, with the exception that GFP positive cells were sorted using the fluorescence associated cell sorter (FACSVantage SE, Becton Dickinson). Acquisition and analysis of the FACS data were performed using the CELLQuest Software (Becton Dickinson). The sorted eGFP positive cells were then tested for their binding capacity to laminin or fibronectin using an adhesion assay.

Briefly, after cell sorting, the GFP positive cells were counted and the density was adjusted to 100 cells/μl in DMEM without FBS. Approximately one thousand cells were spotted per well on a NunClon plate pre-coated with an increasing amount (0, 1, 5 and 10 μg/ml) of laminin or fibronectin. After 2 hrs incubation at 37° C., 5% CO₂, non-attached cells were removed by aspiration, and the wells were washed once with adhesion buffer (PBS +Mg²⁺and Ca²⁺; 2 mM glucose and 1% BSA). Then, the cells were fixed with 3.7% formaldehyde (in PBS) for 10 minutes at room temperature. The fixing solution was removed, and the cells were stained with Crystal violet solution (5% w/v Crystal Violet in 20% v/v ethanol solution) for 5 minutes at room temperature. The cells were then washed extensively with PBS before counting the number of cells attached to each well.

Binding to laminin was impaired in F9 cells cotransfected with dsRNA integrin α3, whereas adhesion to fibronectin was only partially reduced (by about 40%) when compared to the values obtained with F9 wild-type and co-transfection controls. This result demonstrates specificity because α3β1 is the only laminin receptor in these cells but the cells express several fibronectin receptors including α3β1. In addition, dsRNA β1 into F9 cells completely abolished binding to both laminin and fibronectin, since receptors for both molecules contain the β1 subunit. Treatment of cells with dsRNA lacZ had no effect on adhesion of cells to either fibronectin or laminin, providing further evidence of dsRNA specificity.

In summary, these experiments demonstrate that dsRNA-mediated gene silencing is functional and sequence-specific for endogenous genes in embryonic cell lines, thereby allowing specific expression of a target gene to be abolished without interfering with other genes. Although co-transfection of cells with a reporter gene is described here to allow selection of cells transfected with dsRNA, transfection with dsRNA in the absence of reporter is also envisioned.

1. A method of inhibiting expression of a target gene, said method comprising exposing a renewable, mammalian cell to a nucleic acid, said nucleic acid being at least a partially double-stranded ribonucleic acid and having at least 60% sequence identity to a target gene.

2. The method of claim 1, wherein the sequence identity is maintained over at least 18 bases.

3. The method of claim 1, wherein expression of said target gene is inhibited by at least 50%.

4. The method of claim 1, wherein the target gene is a cellular gene.

5. The method of claim 1, wherein the target gene is an endogenous gene.

6. The method of claim 1, wherein the target gene is an oncogene.

7. The method of claim 1, wherein the target gene is a transgene.

8. The method of claim 1, wherein said nucleic acid is introduced into the cell by electroporation.

9. The method of claim 1, wherein said cell is embryonic.

10. The method of claim 1, wherein cell is an embryonic stem cell.

11. The method of claim 1, wherein said cell is a cancer cell.

12. The method of claim 1, further comprising contacting said cell with a protein kinase R (PKR) inhibitor and/or an RNase L inhibitor.

13. The method of claim 12, wherein said protein kinase R inhibitor and/or said RNAse L inhibitor is double stranded RNA specific for PKR and/or RNAseL.

14. The method of claim 1, wherein said ribonucleic acid comprises one strand that is self-complementary.

15. The method of claim 1, wherein said ribonucleic acid comprises two separate complementary strands.

16. The method of claim 1, further comprising synthesis of the nucleic acid outside the cell.

17. The method of claim 1, further comprising synthesis of the nucleic acid inside the cell.

18. The method of claim 17, wherein said nucleic acid is transcribed under the control of an endogenous promoter.

19. The method of claim 17, wherein-said nucleic acid is a product of an expression vector in the cell.

20. The method of claim 19, wherein said expression vector comprises a constitutive promoter operably linked to said nucleic acid.

21. The method of claim 19, wherein said expression vector comprises an inducible promoter operably linked to said nucleic acid.

22. The method of claim 19, wherein said expression vector comprises a tissue-specific promoter operably linked to said nucleic acid.

23. The method of claim 19, wherein said expression vector further comprises sequences encoding PKR and/or RNAse L dsRNA operably linked to a promoter.

24. The method of claim 19, wherein said expression vector further comprises a promoter operably linked to a reporter gene, wherein said promoter is selected from the group consisting of a second promoter, a bi-directional promoter or a promoter driving a polycistronic message.

25. The method of claim 24, wherein said reporter gene is selected from the group consisting of a fluorescent protein, an antibiotic, beta- galactosidase and iuciferase.

26. The method of claim 24, further comprising selecting cells expressing said reporter gene for expansion in cell culture.

27. The method of claim 26, wherein said cells are embryonic stem cells.

28. The method of claim 1, in which the cell is present in an organism and inhibition of target gene expression demonstrates a loss-of function phenotype.

29. An expression construct comprising a nucleic acid encoding at least a partially double-stranded ribonucleic acid having at least 60% sequence identity to a target gene of interest and a second nucleic acid encoding at least one dsRNA molecule effective in inhibiting one or more of PKR, RNAseL, 2′,5′ oligoadenylate cyclase and Mx protein.

30. A kit comprising reagents for inhibiting expression of a target gene in a cell, wherein said kit comprises a means for introduction of a ribonucleic acid (RNA) into a mammalian cell in an amount sufficient to inhibit expression of the target gene, and wherein the RNA has at least a partialily partially double- stranded structure with an identical nucleotide sequence as compared to a portion of the target gene.

31. A mammalian cell exhibiting gene silencing achieved by the method of claim 1.

32. A transgenic mammal, other than human, exhibiting targeted gene silencing produced using at least one cell according to claim 31.

33. A method for treating a disease comprising administering to a subject embryonic stem cells obtained by the method of claim 10.

34. A method of treating a disease comprising administering an effective amount of the construct of claim 29.

35. A method of assigning function to a DNA sequence, said method comprising:
a) exposing a mammalian cell to a nucleic acid, said nucleic acid being at least a partially double-stranded ribonucleic acid and having at least 60% sequence identity to a desired DNA sequence of unassigned function, in an amount sufficient to inhibit gene expression of the cellular homologue of said desired DNA sequence,
b) identifying a phenotype of said mammalian cell compared to wild type, and
c) assigning said phenotype to said desired nucleic acid.

36. A method according to claim 35, wherein said desired nucleic acid is cloned into an expression vector to allow the production of a transcript that is self-complementary.

37. A method of identifying DNA responsible for conferring a particular phenotype in a cell which method comprises
a) constructing a cDNA or genomic library of the DNA of said cell in a suitable vector in an orientation relative to a promoter(s) capable of initiating transcription of said cDNA or DNA to double stranded (as) RNA upon binding of an appropriate transcription factor to said promoter(s),
b) introducing said library into one or more of mammalian cells comprising said transcription factor, and
c) identifying and isolating a particular phenotype of said cell comprising said library and identifying the DNA or cDNA fragment from said library responsible for conferring said phenotype.
